(19) United States
(12) Patent Application Publication
(10) Pub. No.: US 2024/0350039 A1
(43) Pub. Date: Oct. 24, 2024

(54) SYSTEMS AND METHODS FOR CONDUCTING AUDIOMETRIC TESTING WITHIN A HEARING CONVERSATION PROGRAM

(71) Applicant: Examinetics, Inc., Overland Park, KS (US)

(72) Inventors: Matthew Peter Kafer, Belton, MO (US); Cassandra Lynn Ford, Kansas City, MO (US); Edward Henry Stratemeier, IV, Prairie Village, KS (US)

(21) Appl. No.: 18/630,874

(22) Filed: Apr. 9, 2024

Related U.S. Application Data
(63) Continuation-in-part of application No. 18/137,375, filed on Apr. 20, 2023.

(51) Int. Cl. A61B 5/12; A61B 5/00; G09B 5/02; G09B 19/00; G16H 40/67

(52) U.S. Cl. CPC A61B 5/123; A61B 5/0022; A61B 5/6803; G09B 5/02; G09B 19/00; G16H 40/67; A61B 2560/0228; A61B 2560/0247

(57) ABSTRACT

A computer-implemented method for audiometric includes displaying, on a mobile device, a training video to a subject to be tested; conducting an audiometric test of the subject using the mobile device to control an audiometer in wireless communication with the mobile device, wherein the audiometer is separate from the mobile device and includes a tone generator in electronic communication with a headset worn by the subject; displaying, on the mobile device, results of the audiometric test; electronically comparing the results of the audiometric test with a baseline test for the subject; displaying, on the mobile device, a notification of a hearing shift if the results of the audiometric test differ from the baseline test by more than a predetermined threshold; and receiving, via the mobile device, an electronic acknowledgment from the subject that the subject has viewed the training video, the results of the audiometric test, and the notification of the hearing shift, if any.

### CROSS-REFERENCE TO RELATED APPLICATIONS

This application is a continuation-in-part of U.S. patent application Ser. No. 18/137,375, filed Apr. 20, 2023, for SYSTEMS AND METHODS FOR CONDUCTING AND VALIDATING AN AUDIOMETRIC TEST, which is incorporated herein by reference in its entirety.

### TECHNICAL FIELD

The present disclosure relates to audiometric testing and, more particularly, to systems and methods for performing audiometric testing within a hearing conservation program.

### BACKGROUND

Noise, or unwanted sound, is a significant occupational health problem. Sound consists of pressure changes in a medium (usually air), caused by vibration or turbulence. These pressure changes produce waves emanating away from the turbulent or vibrating source. Exposure to high levels of noise causes hearing loss and may result in other harmful health effects. The extent of hearing damage depends primarily on the intensity of the noise and the duration of the exposure.

Noise-induced hearing loss can be temporary or permanent. Temporary hearing loss results from short-term exposures to noise, with normal hearing returning after a period of rest. Generally, prolonged exposure to high noise levels over a period of time gradually causes permanent damage.

To reduce the likelihood of hearing loss, various regulatory bodies and industry authorities have established standards for employees subjected to high levels of noise at work. Such standards typically involve periodic audiometric (i.e., hearing) testing. For example, one set of regulations requires employers to monitor the hearing of all employees whose noise exposure is equivalent to or greater than the noise exposure received in 8 hours where the noise level is constantly 85 dB.

The specific means for establishing a hearing conservation program is typically left to the employer's discretion. This puts employers of all sizes in the difficult position of developing and administering in-house programs that satisfy a myriad of requirements with severe consequences for non-compliance.

As only one example of the difficulties of establishing a hearing conservation program, the devices used to conduct audiometric tests, i.e., audiometers, can be expensive and difficult to transport. For example, a typical audiometer may cost thousands of dollars and be too heavy or bulky to easily move between testing sites, forcing those desiring an audiometric test to travel to the fixed location of the audiometer. This is inconvenient and unsuitable to on-site testing, such as testing employees to comply with Occupational Safety and Health Administration (OSHA) regulations.

Furthermore, even expensive audiometers may produce invalid results. For example, background noise can make it difficult for a subject to hear tones produced by the audiometer. Moreover, the audiometer and/or a headset used by the audiometer can become uncalibrated, producing tones that diverge in frequency and/or sound pressure levels from standard test tones. In addition, subjects of an audiometric test may not be properly instructed on taking the test, resulting in user error and preventing hearing threshold levels from being accurately determined. Invalid tests cannot be used to satisfy the regulations of OSHA or other organizations and may result in adverse consequences for an employer.

What is needed, therefore, is a system and method for conducting audiometric testing within a hearing conservation program that ensures valid tests and reduces the risk of non-compliance with established standards.

### SUMMARY OF THE DISCLOSURE

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

According to one aspect, a system for audiometric testing comprises an audiometer including a tone generator in electronic communication with a headset worn by a subject to be tested. The system also includes a mobile device in electronic communication with the audiometer, where the audiometer and the mobile device are separate devices, and the mobile device includes a display screen, one or more processors, and a non-non-transitory machine-readable medium including program code that causes the one or more processors to perform a method for audiometric testing. The method includes displaying, on the display screen, a training video to the subject. The method also includes conducting an audiometric test of the subject using the mobile device to control the audiometer. The method further includes displaying, on the display screen, results of the audiometric test. In addition, the method includes electronically comparing the results of the audiometric test with a baseline test for the subject. Furthermore, the method includes displaying, on the display screen, a notification of a hearing shift if the results of the audiometric test differ from the baseline test by more than a predetermined threshold. The method also includes receiving, via the mobile device, an electronic acknowledgment from the subject that the subject has viewed the training video, the results of the audiometric test, and the notification of the hearing shift, if any.

In another aspect, a computer-implemented method includes displaying, on a mobile device, a training video to a subject to be tested. The method also includes conducting an audiometric test of the subject using the mobile device to control an audiometer in wireless communication with the mobile device, wherein the audiometer is separate from the mobile device and includes a tone generator in electronic communication with a headset worn by the subject. The method further includes displaying, on the mobile device, results of the audiometric test. In addition, the method includes electronically comparing the results of the audiometric test with a baseline test for the subject. Furthermore, the method includes displaying, on the mobile device, a notification of a hearing shift if the results of the audiometric test differ from the baseline test by more than a predetermined threshold. The method also includes receiving, via the mobile device, an electronic acknowledgment from the subject that the subject has viewed the training video, the results of the audiometric test, and the notification of the hearing shift, if any.

### BRIEF DESCRIPTION OF THE DRAWINGS

The accompanying figures are provided by way of illustration and not by way of limitation. The foregoing aspects and other features of the disclosure are explained in the following description, taken in connection with the accompanying example figures relating to one or more embodiments, in which:

FIG. 1 is a schematic diagram of a system for conducting audiometric testing within a hearing conservation program.

FIG. 2 is a schematic diagram of software modules within a memory of a mobile device.

FIG. 3A is a flow chart of a method for conducting audiometric testing within a hearing conservation program.

FIG. 3B and FIG. 3C are user interfaces for registering a subject to be tested.

FIG. 3D is a user interface for training the subject.

FIG. 3E is a user interface and data flow diagram for testing the subject.

FIG. 3F is a report of the results of an audiometric test.

FIG. 3G is a report of a hearing shift.

FIG. 3H is a report of multiple employees identified with hearing shifts.

FIG. 4A is a flowchart of a method for validating an audiometric test.

FIG. 4B is a user interface for training a test administrator.

FIG. 5A is a schematic diagram of a system for detecting ambient noise.

FIG. 5B is a user interface for validating a room.

FIG. 6A is a schematic diagram of a calibrator.

FIG. 6B is a user interface for equipment validation.

### DETAILED DESCRIPTION

In the following Detailed Description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

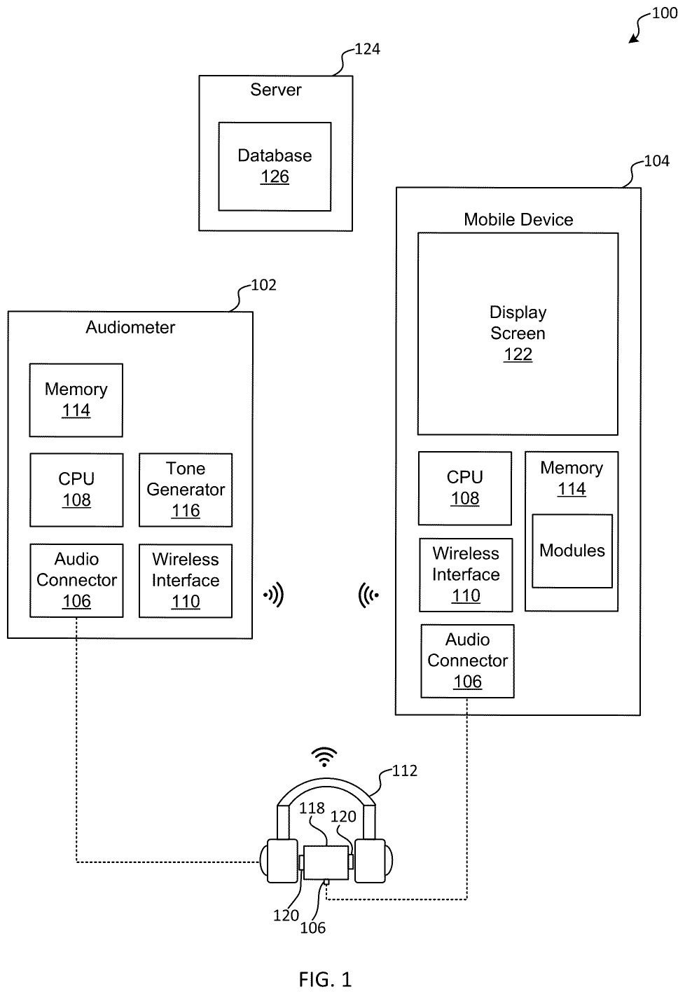

FIG. 1 is a schematic diagram of a system 100 for conducting audiometric testing within a hearing conservation program according to an embodiment. The system 100 may include an audiometer 102 and a mobile device 104 for controlling the audiometer 102. As illustrated, the audiometer 102 and the mobile device 104 may be separate devices that are in electronic communication via a wireless or wired connection.

The audiometer 102 may include one or more audio connectors 106, which may be embodied as physical audio ports/jacks, as well as audio circuitry, e.g., Analog-to-Digital Converters (ADCs), Digital-to-Analog Converters (DACs), filters, or the like, for transferring audio data to and from a CPU 108. Alternatively, or in addition, the audiometer 102 may include a wireless interface 110 for transmitting and receiving data (e.g., audio, messages) to and from the mobile device 104 and/or a headset 112, which may be coupled via a wired connection to an audio connector 106 in some embodiments. The wireless interface 110 may implement one or more wireless standards, including, without limitation, Bluetooth, Wi-Fi (802.11), ZigBee, and/or Z-Wave.

The headset 112 may be embodied as headphones, earbuds, or the like, which include a pair of small loudspeaker drivers worn on or around the head over a user's ears. The drivers are electroacoustic transducers that convert an electrical signal to a corresponding sound. Suitable headsets for conducting an audiometric test include, without limitation, a DD65v2 headset, available from RadioEar.

The CPU 108 may be any suitable microprocessor, microcontroller, Digital Signal Processor (DSP), Field Programmable Gate Array (FPGA) or other device that may execute, for example, instructions stored in a memory 114. In various embodiments, the memory 114 is implemented using any suitable combination of Random Access Memory (RAM), Read-Only Memory (ROM), Electrically Erasable Programmable Read-Only Memory (EEPROM), or the like.

In one embodiment, the audiometer 102 includes a tone generator 116 that generates, for example, standardized tones to play on the headset 112. The tone generator 116 may include, without limitation, a 32-bit DAC, one or more filters, and/or other circuitry needed for generating analog audio output via the audio connector 106. A suitable tone generator 116 may include, for example, an ES9218 DAC available from EES in combination with an STM32 processor available from STMicroelectronics. In some embodiments, including fully wireless embodiments, the functionality of the tone generator 116 may be incorporated into the CPU 108. However, for clarity of description, the tone generator 116 is illustrated as a separate component.

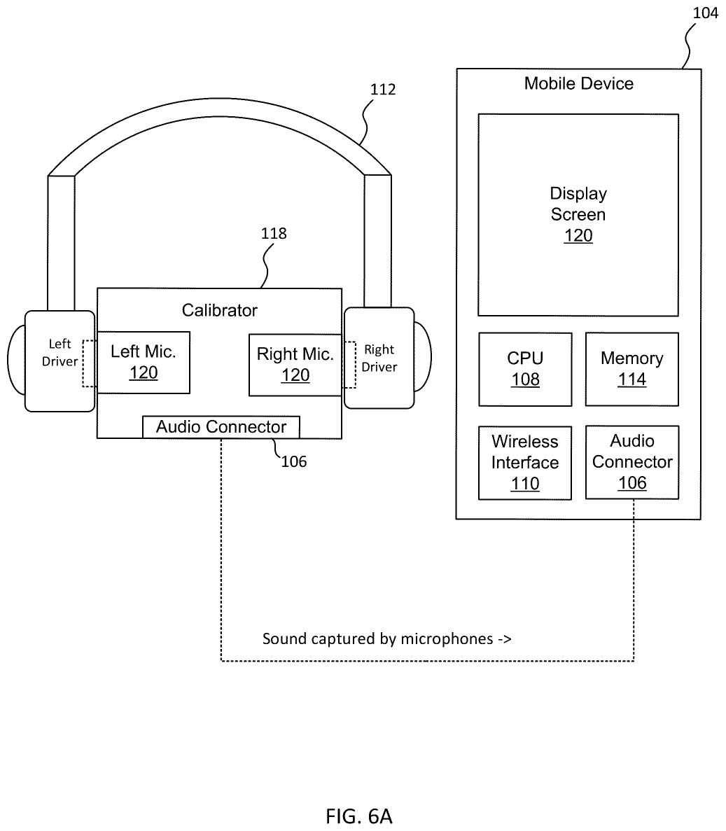

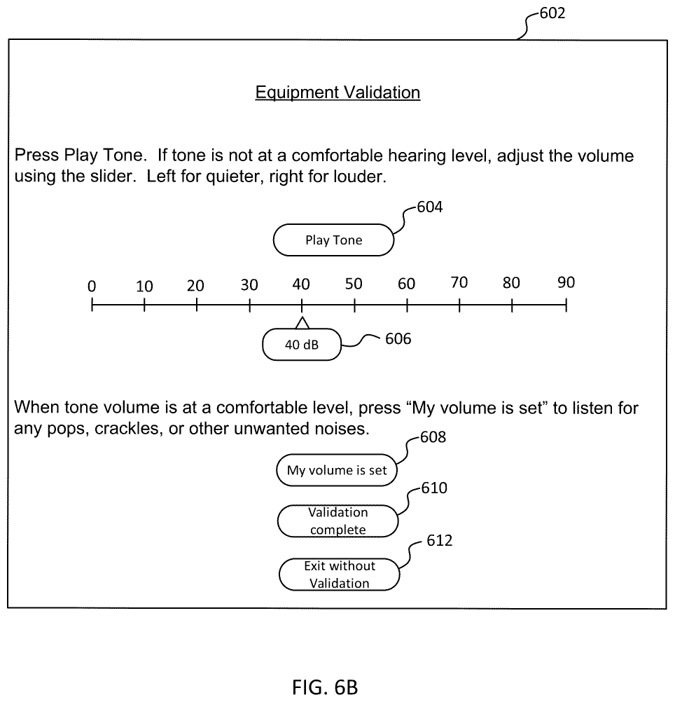

In some embodiments, the headset 112 and the audiometer 102 may be calibrated using a calibrator 118, as described in greater detail in connection with FIG. 6A. The calibrator 118 may include a housing having a width that approximates a distance between a person's ears. Further, the calibrator 118 may include one or more microphones 120. Suitable microphones 120 may include, without limitation, a I437L microphone, available from MicW.

In the illustrated embodiment, two microphones 120 are provided on opposite sides of the housing in order to be placed in proximity to the left and right loudspeaker drivers of the headset 112. The calibrator 118 may also include an audio connector 106 for electrically connecting the microphones 120 to an audio connector 106 within the mobile device 104. Wireless communication may be used in other embodiments. In other embodiments, a single microphone could be used.

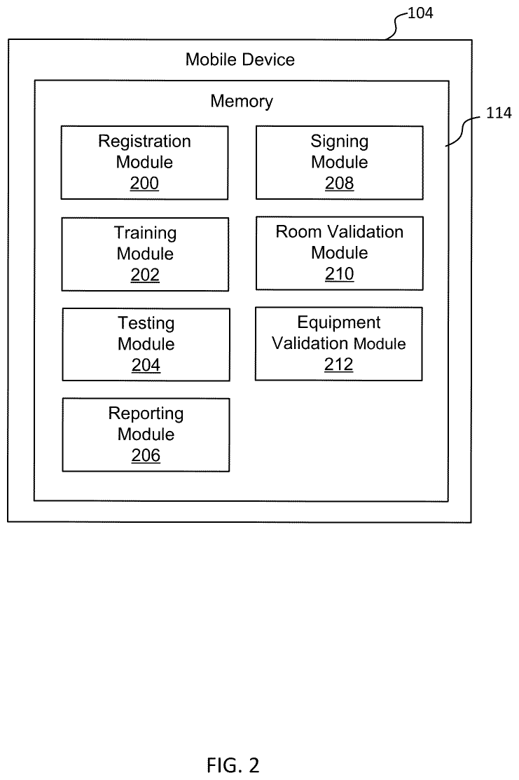

The mobile device 104 may also include various components found in the audiometer 102, such as, for example, a wireless interface 110, a CPU 108, and a memory 114, each of which may be the same as, or different from, the corresponding components in the audiometer 102. The memory 114 may store various software modules as described in greater detail with respect to FIG. 2. The mobile device 104 may further include a display screen 122, such as a Liquid Crystal Display (LCD), a Light-Emitting Diode (LED) display, an Organic Light-Emitted Diode (OLED) display, or the like. In some embodiments, the mobile device 104 may be implemented using a tablet computer, such as an iPad® tablet, available from Apple Corporation.

While FIG. 1 illustrates a single audiometer 102 and a single mobile device 104, those of skill in the art will recognize that multiple audiometers 102 and mobile devices 104 may be used within the system 100. For example, certain aspects of the present disclosure may be performed using one mobile device 104, while other aspects are performed by a different mobile device 104. Furthermore, the mobile devices 104 need not be identical.

The system 100 may further include a server 124, which may be a computer system configured to serve data to the mobile device 104 via the wireless interface 110 over a computer network, such as a Local Area Network (LAN) and/or a Wide Area Network (WAN), such as the Internet. The data may be stored within the server 124 in a database 126, which may be a relational database, e.g., structured query language (SQL) database, a hierarchical database, an object-oriented database, or the like. The database 126 may be implemented using various storage devices, such as hard disk drives (HDDs) and/or solid-state drives (SSDs). In some embodiments, the database 126 may be implemented using cloud storage, such as Amazon S3® or AWS®. As described in greater detail below, the database 126 may be used to store employee data, including personally identifiable information (PII), audiometric test data, signed audiometric reports, and the like.

Referring to FIG. 2, the memory 114 of the mobile device 104 may store a number of software modules containing instructions that cause the CPU 108 and other hardware components to perform the various methods disclosed herein. For example, the memory 114 may store, without limitation, a registration module 200, training module 202, a testing module 204, a reporting module 206, a signing module 208, a room validation module 210, and an equipment validation module 212. Those of skill in the art will recognize that the functionality of various modules may be combined and/or additional modules may be included without departing from the scope of the disclosure. In other words, the software architecture disclosed herein is only one example of possible organizations of instructions to perform the disclosed methods.

The registration module 200 identifies the subject (e.g., employee) to be tested, either by looking up the subject in the database 126 based on personally identifiable information (PII), or by obtaining various registration information from the subject. Information received by the registration module 200 may be stored in the database 126 for subsequent retrieval and reporting.

The training module 202 displays a training video to the subject regarding different aspects of hearing protection, as well as the audiometric testing procedures. The training video may be displayed, for example, on the display screen 122 of the mobile device 104 (e.g., tablet). In some embodiments, the subject must view the training video in order for the audiometric test to proceed. As such, the training module 202 may interact with the testing module 204 to prevent the audiometric test from taking place until playback of the training video is complete and/or the subject has acknowledged watching the training video.

The testing module 204 conducts the audiometric test of the subject. In some embodiments, the audiometric test is performed according to the Hughson-Westlake procedure using the audiometer 102 under control of the mobile device 104, as explained in greater detail below.

The reporting module 206 displays, on the mobile device 104, the results of the audiometric test, which may include an audiogram that provides, for each ear of the subject, a graph of hearing threshold levels of the subject for a set of frequencies. Each hearing threshold level may include an indication of the sound pressure level at which the subject indicated that the tone at a given frequency was heard within a predetermined time interval. According to the Hughson-Westlake procedure, a hearing threshold level is defined as the lowest decibel hearing level at which responses occur in at least one half of a series of ascending trials. The minimum number of responses according to this procedure needed to determine the threshold of hearing is two responses out of three presentations at a single level.

In some embodiments, the reporting module 206 compares the results of the audiometric test with a baseline test for the subject (e.g., stored in the database 126) and displays a notification of a hearing shift if the results of the audiometric test differ from the baseline test by more than a predetermined threshold (e.g., 10 dB). The reporting module 206 may also generate an spoken explanation of the results using text-to-speech synthesis.

The signing module 208 receives, via the mobile device 104, an electronic acknowledgment from the subject that the subject has, without limitation, viewed the training video, the results of the audiometric test, and the notification of the hearing shift, if any. The electronic acknowledgment may be in the form of an electronic signature that is applied to a report containing the audiometric test and/or the notification of the hearing shift. The signed report may be stored in the database 126 for later retrieval.

In some embodiments, testing module 204 may be prevented from starting the audiometric test until certain validation steps are completed, typically by a test administrator. For example, the audiometric test may not proceed until (1) the room in which the audiometric test is to be administered has a set of background sound pressure levels that conform to a predetermined standard, as determined by the room validation module 210, and (2) the calibration of the audiometer 102 and associated headset 112 has been validated at least once on a day (or other time period) in which the audiometric test is to be administered, as determined by the equipment validation module 214. In some embodiments, the equipment validation module 214 may also facilitate calibration of the audiometer 102 and headset 112 if needed.

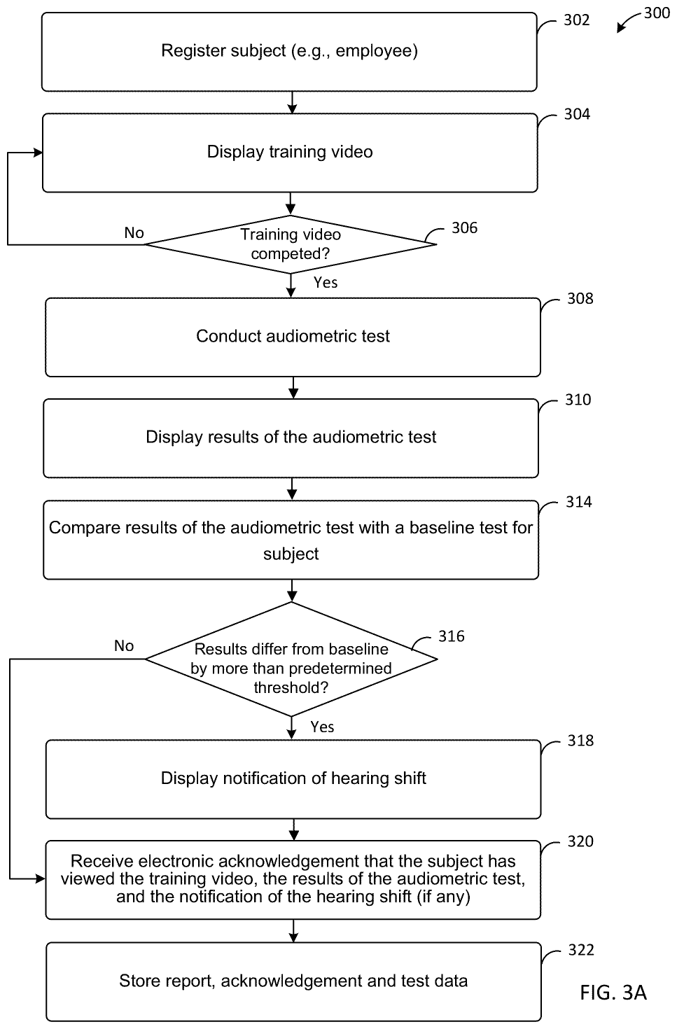

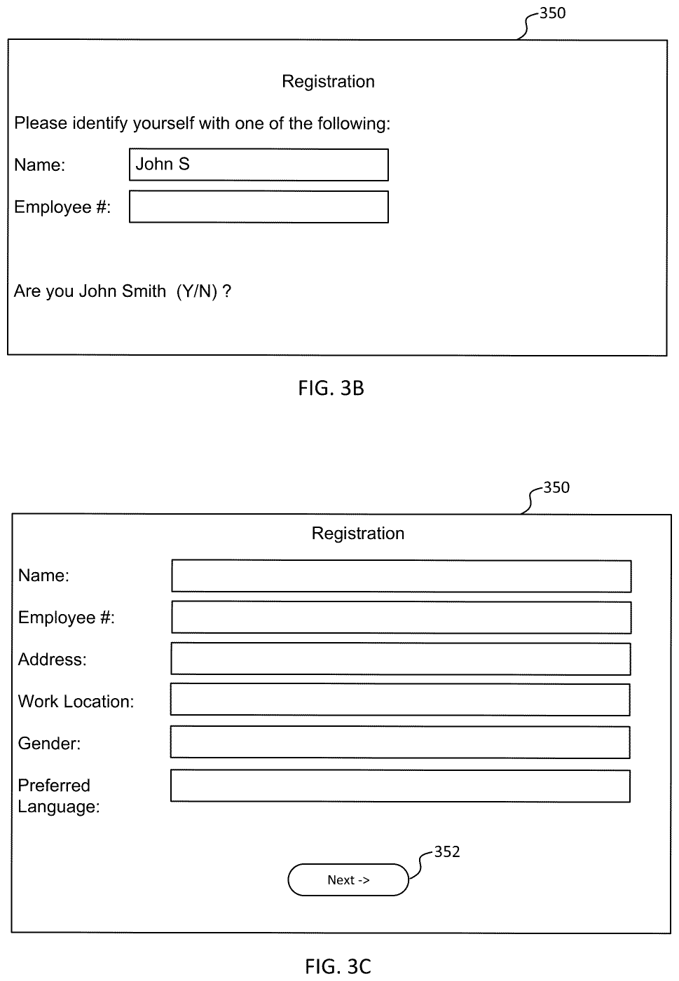

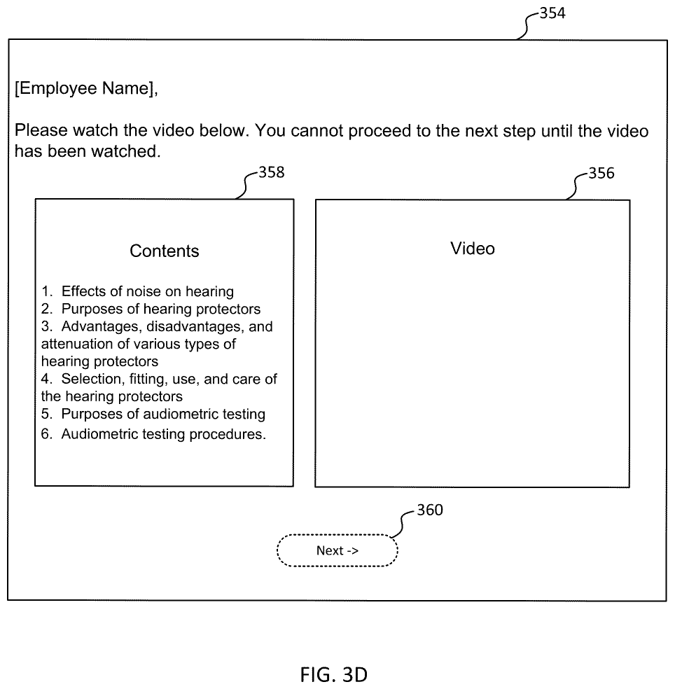

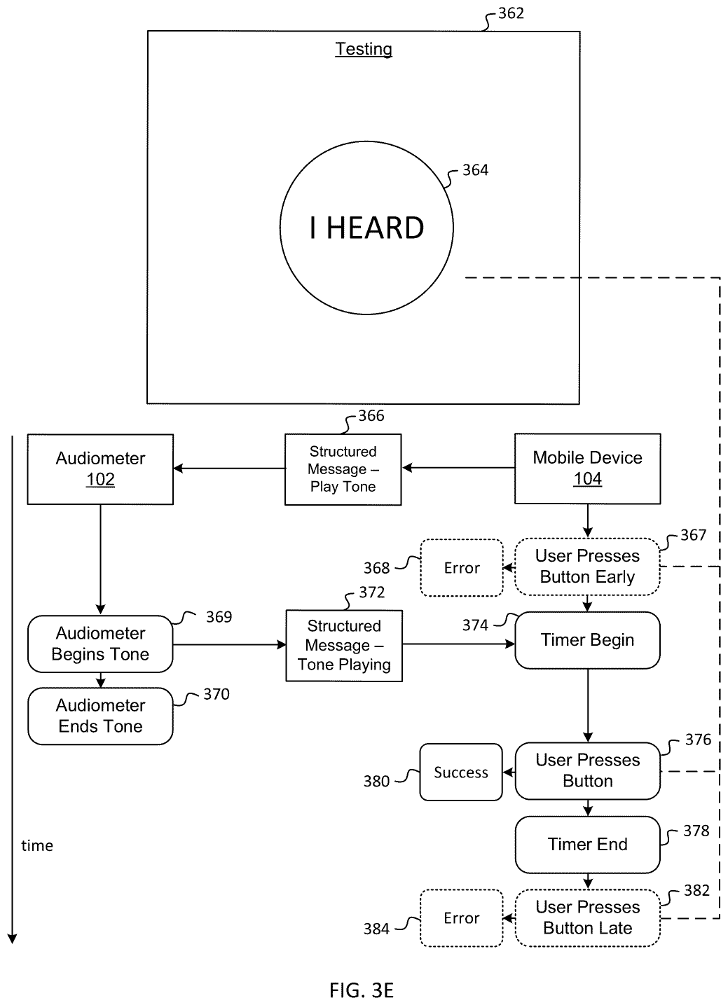

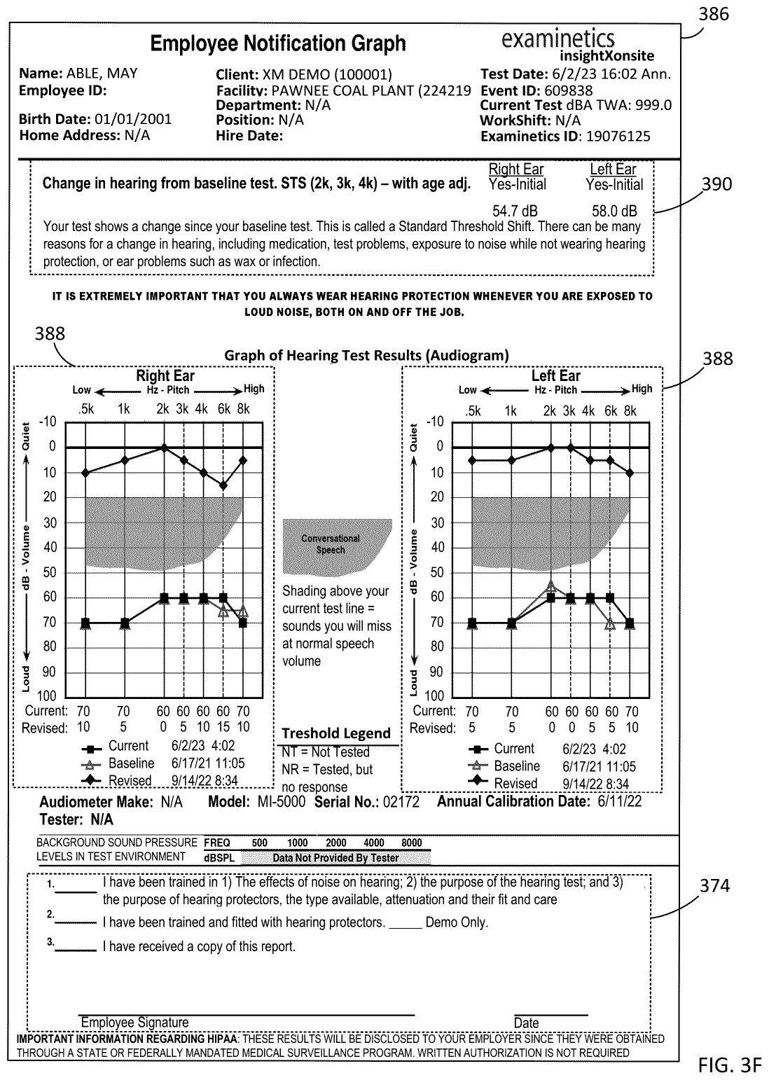

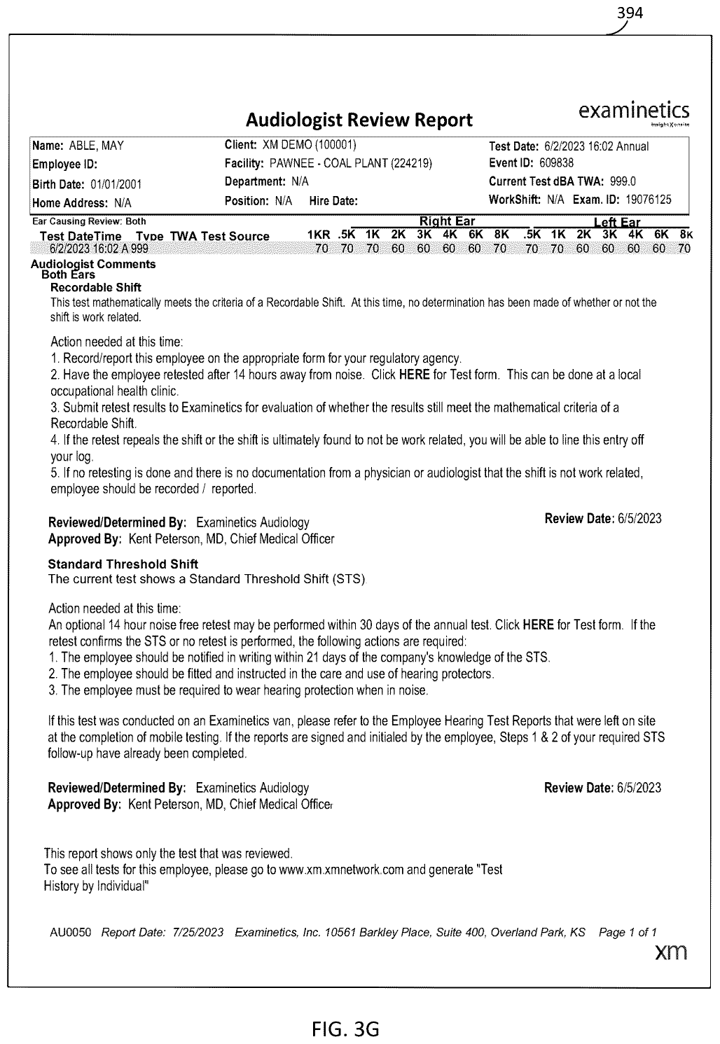

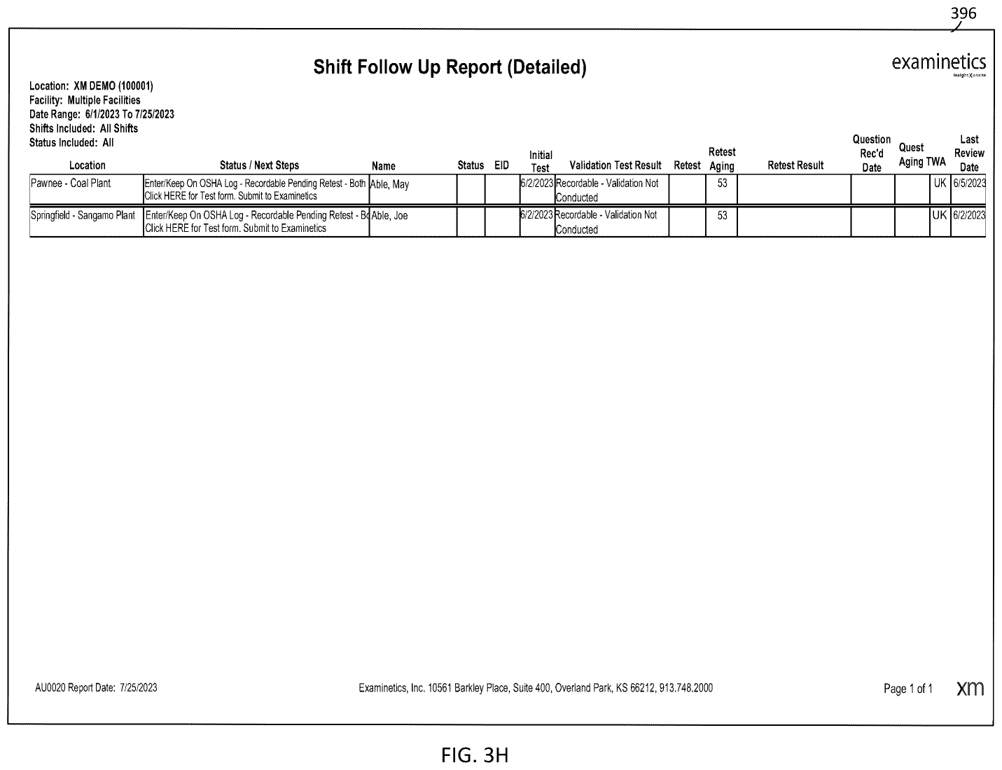

FIG. 3A is a flowchart of a method 300 for conducting an audiometric test according to some embodiments of the present disclosure. The method 300 may be performed by the mobile device 104 in conjunction with the audiometer 102, the server 124, the database 126, and other components of the system 100, as discussed in connection with FIG. 1.

The method 300 begins, in one embodiment, by registering 302 the subject to be tested. For example, as shown in FIG. 3B, a user interface 350 (which may be generated by the registration module 200) asks the subject to identify themselves with some personally identifiable information (PII), such as a name or employee number. The mobile device 104 may be configured to search the database 126 in response to the subject providing all or some of the requested PII and prompt the subject whether they are a particular individual in the database 126 matching the provided information. In other embodiments, the subject may need to provide their full name or employee number and affirmatively press a “search” button (not shown).

As shown in FIG. 3C, if the subject is not found in the database 126, the user interface 350 may prompt the subject to provide various information, such as their name, employee number, address, work location, gender, preferred language, and the like. Some fields, such as work location and gender, may be selected from a pre-populated list of options. When the subject has finished providing the requested information, they may select a “next” button 352 or the like.

With continuing reference to FIG. 3A, the method 300 may proceed by displaying 304 a training video to the subject via the display screen 122 of the mobile device 104. The training video may include various information relating to a hearing conservation program, non-limiting examples of which include:
1. Effects of noise on hearing,
2. Purposes of hearing protectors;
3. Advantages, disadvantages, and attenuation of various types of hearing protectors;
4. Selection, fitting, use, and care of the hearing protectors;
5. Purposes of audiometric testing, and
6. Audiometric testing procedures.

For example, in connection the audiometric testing procedures, the instructions to the subject may include the following:
1. You will hear a series of tones during your test. As soon as you hear a tone, tap the “I Heard” button on your device.
2. Some of the tones you will hear will be very quiet. Please tap the “I Heard” button if you just barely hear the tone.
3. Missing a tone will not impact the outcome of the test. If you are distracted by a brief noise, don't worry. You will have more than one opportunity to respond to each tone.
4. After completion of your test, you will be shown a graph and hear an explanation of your test results.
5. Please remove hearing aids, glasses, earrings, or anything else you may be wearing between your head and the headphones, then place the headphones back over your ears.
6. When you are finished, click the “Finished” button.

Of course, other instructions could be used within the scope of the present disclosure. In some configurations, the instructions may be updated as needed.

FIG. 3D illustrates a user interface 354, which may be generated by the training module 202, for displaying the training video. The user interface 354 may include a video window 356 where the training video is to be displayed. In some embodiments, the user interface 354 may additionally include a text window 358 for displaying text, such as step-by-step written instructions and/or a table of contents of the training video. In the latter case, the subject may be able to review different portions of the training video by selecting individual items of the table of contents shown in the text window 358.

In some embodiments, the subject will not be able to proceed with testing until the entire video has been watched. For example, as shown in FIG. 3A, a determination 306 may be made whether the training video has been completed. If not, the method 300 may return to step 304 to play (or replay) the training video to completion.

In certain embodiments, the training video cannot be skipped, but only paused or rewound to ensure that the subject watches the entire training video. In still other embodiments, the mobile device 104 may employ visual attention and focus tracking technology in order to ensure that the subject is paying attention to the training video. Such technology may include, without limitation, eye/focus tracking via a front-facing camera on the mobile device 104 to determine whether the subject is watching the screen while the video is being presented. In some embodiments, the user may be quizzed on aspects of the training video before being allowed to continue. After the training video is complete, the user may press a “next” button 360 or the like, which may be deactivated (grayed-out) until the training video has played and/or all other requirements in connection with the training video have been completed.

Returning to FIG. 3A, the method 300 continues by conducting 308 the audiometric test. In some embodiments, the testing module 204 generates a user interface 362 for display on the display screen 122, as shown in FIG. 3E. For simplicity, the testing interface 362 may include a single button 364 or other central or prominent control indicating that the user heard the generated tone.

FIG. 3E also includes a dataflow and timing diagram showing the timing of messages between the mobile device 104 and the audiometer 102. Initially, the mobile device 104 sends a first structured message 366 instructing the audiometer 102 to play a tone of a certain frequency and a certain decibel level in particular ear of the subject. For example, the first structured message 366 may include a Bluetooth command or message (packet) and may instruct the audiometer 102 to pay a 1000 Hz tone at 60 dB in the subject's left ear (i.e., in the left driver of the headset 112).

If the user presses the button early (i.e., before the tone is played) at point 367, an error is registered at point 368.

Otherwise, the audiometer 102 begins the tone at point 369 and ends the tone at point 370. The audiometer 102 sends a second structured message 372 (e.g., Bluetooth packet) to the mobile device 104 indicating that the tone is playing. The second structured message 372 may be sent when the audiometer 102 begins playing at point 369 (as illustrated) or, in another embodiment, when the audiometer 102 ends the tone at point 370 (not shown), indicating that the tone has been played.

When the second structured message 372 is received, the mobile device 104 begins a timer at point 374. The timer may proceed for a predetermined amount of time, e.g., 400 milliseconds.

If the subject presses the button 364 at point 376 before the timer ends at point 378, a success is registered at point 380. If, however, the subject presses the button 364 late at point 382 after the timer ends at point 378, an error is registered at point 384.

As previously noted, the test may be conducted according to the Hughson-Westlake procedure, in which the threshold of hearing is defined as 2 out of 3 (or 3 out of 5) correct responses (successes) during the ascending portion of the tone presentation. If the patient responds when the tone is ascending, the test will automatically decrease the level by 10 dB. The patient has to respond to the same intensity 2 out of 3 or 3 out of 5 times for the threshold to be recorded. Intensity increases are in steps of 5 dB while the intensity decreases are in steps of 10 dB in one embodiment.

The time between tones (and, hence, messages 366 from the mobile device 104) may vary within a particular range (e.g., 720 to 2000 milliseconds) in order to prevent the subject from anticipating the timing of the tones. As such, messages 366 from the mobile device 104 may be delayed or timed within the particular range by a random or pseudo-random factor.

Multiple tones are generated in each ear of the subject using a series of frequencies, e.g., 500 Hz, 1000 Hz, 2000 Hz, 3000 Hz, 4000 Hz, and 8000 Hz. In effect, when the subject fails to indicate that the particular tone has been heard within a predetermined time interval, the sound pressure level for the missed tone will be progressively increased (although not necessarily in a sequential fashion) until the subject has indicated that the particular tone has been heard within the predetermined time interval. In other words, the audiometric test determines how loud sounds need to be at different frequencies in order for the subject to hear them.

Using the foregoing procedure, it is nevertheless possible that user responses may result in an invalid test. For example, the subject may provide inconsistent responses or too frequently “hear” tones that have not been played. However, assuming that hearing threshold levels can be determined, the method 300 continues, as shown in FIG. 3A, by displaying 310 the results of the audiometric test.

Referring to FIG. 3F, the results, which may be generated by the reporting module 206, may be in the form of a hearing report 386, which may be displayed on the display screen 122 of the mobile device 104 and/or be printed on paper. The hearing report 386 may include audiograms 388 for the right and left ears, respectively.

Returning to FIG. 3A, the method 300 may continue, in one embodiment, by comparing 314 (via the reporting module 206) the results of the audiometric test with a baseline test for the subject, if any. The baseline test for the subject may be the last test taken by the subject, the results for which were previously stored in the database 126. The baseline test may not always be the first test for an employee at the company. For example, if there was a significant change of hearing, and if that change was subsequently confirmed by a retest, then the new test would become the baseline for subsequent comparison, even though the database 126 may contain several years of test results for the employee.

Returning to FIG. 3A, a determination 316 is made whether the test results differ from the baseline test by more than a predetermined threshold (e.g., 10 dB). If not, the method continues at step 320. If so, the method 300 continues by displaying 318 a notification 390 of a hearing shift, an example of which is shown in FIG. 3F. In some embodiments, the notification 390 may be displayed with the audiograms 388 in the same hearing report 386. In other embodiments, the notification 390 may be displayed in a separate report and be separately read and acknowledged by the subject.

As shown in FIG. 3A, the method 300 continues by receiving 320 an electronic acknowledgement that the subject has viewed the training video, the results of the audiometric test, and the notification of the hearing shift, if any. The electronic acknowledgement may be in the form of a signature electronically captured via the mobile device 103 in response to the subject signing their name with a finger or stylus. For example, the hearing report 386 shown in FIG. 3F, in addition to the audiograms 388 and any notifications 390 of a hearing shift, may further include an acknowledgement section 392, which may have a space for the subject's signature, a date of signature, and/or lines for the subject to initial indicating at least that the subject has been trained and received a copy of the report. The subject's signature may be applied to and/or stored with the report, such that subsequent retrieval of the report will include the signature. The hearing report 386 may be embodied, in some implementations, as a portable document format (PDF) document using the signature feature of Adobe Acrobat®.

After receipt of the electronic acknowledgement, the method 300 may conclude by storing 322 the report 386, the acknowledgement, and any associated test data in the database 126. Such data may be used for auditing purposes (i.e., to demonstrate that any applicable standards have been satisfied) and/or for reporting purposes.

An example of another report 394 is as shown FIG. 3G, which may be provided to an employer in response to detecting a hearing shift for an employee. The report may include action items to be performed and may be reviewed and acknowledged by a company audiologist or other healthcare professional. The report 394 may be updated whenever it is reviewed and after retesting of the subject. Thus, the report 394 may be a continuing record of a particular hearing shift of an employee and all efforts to document and mitigate it.

As shown in FIG. 3H, another report 396 may include a list of employees, or employees at a particular location, that have experienced hearing shifts during a particular time period, allowing an employer to track the status of each such employee, including retesting. A wide variety of reports are possible using the stored data in the database 126.

As previously noted, ensuring the validity of an audiometric test can be challenging. For example, background noise can make it difficult for a subject to hear tones produced by the audiometer 102. Moreover, the audiometer 102 and/or a headset used 112 by the audiometer can become uncalibrated, producing tones that diverge in frequency and/or sound pressure levels from standard test tones. Invalid tests cannot be used to satisfy regulations by OSHA and other organizations.

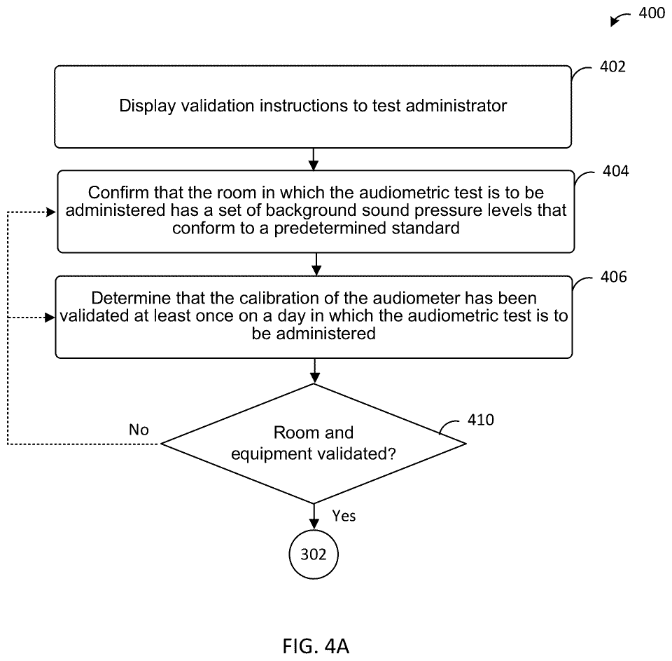

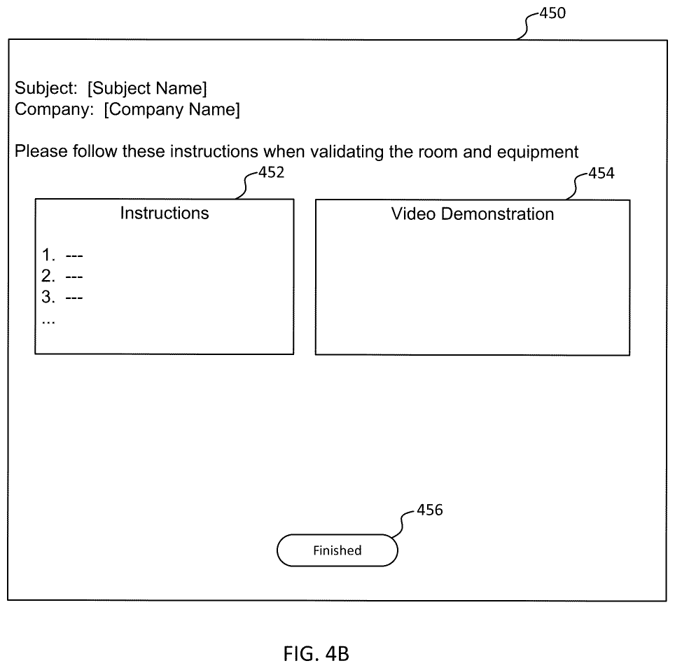

In some embodiments, a number of steps are performed prior to the method 300 of FIG. 3A in order to ensure the validity of the audiometric test. For example, FIG. 4A is a flow chart of a method 400 for conducting a valid audiometric test. The method 400 may be performed using the audiometer 102 and the mobile device 104 in conjunction with a test administrator rather than the subject of the test.

The method 400 begins by displaying 402 validation instructions to the test administrator. The validation instructions may include instructions for validating the room in which the audiometric test is to be administered, as well as instructions for validating and/or calibrating the equipment used in the audiometric test. The validation instructions may be displayed automatically (or periodically) or in response to activation of a particular control (not shown).

FIG. 4B illustrates an example user interface 450, which may be provided by the training module 202, for displaying instructions 452 to the test administrator. The user interface 450 may include a video demonstration area 454, which may include a video presentation depicting step-by-step instructions to the test administrator for how to connect various equipment, such as the calibrator 118, as well as to perform the validation of the room and test equipment. In addition, the user interface 450 may include a control 456 by which the test administrator may acknowledge that they have reviewed the instructions 452 and/or video demonstration 454. In one embodiment, activation of the control 456 is a prerequisite for the subject to be able to subsequently take the audiometric test.

Returning to FIG. 4A, the method 400 continues by confirming 404 that a room in which the audiometric test is to be administered has a set of background sound pressure levels that conform to a predetermined standard. An example standard for the set of background pressure levels is shown below in Table 1. As illustrated, a plurality of octave-band center frequencies (measured in Hertz) may have corresponding maximum sound pressure levels (measured in dB). For example, in order for the room to be validated, at 500 Hz, the maximum sound pressure level is 40 dB, while at 2000 Hz, the maximum sound pressure level is 47 dB.

TABLE 1

| | | | | | |
|---|---|---|---|---|---|
| Octave-band | 500 | 1000 | 2000 | 4000 | 8000 |
| center | | | | | |
| frequency (Hz) | | | | | |
| Maximum | 40 | 40 | 47 | 57 | 62 |
| sound | | | | | |
| pressure level | | | | | |
| (dB) | | | | | |

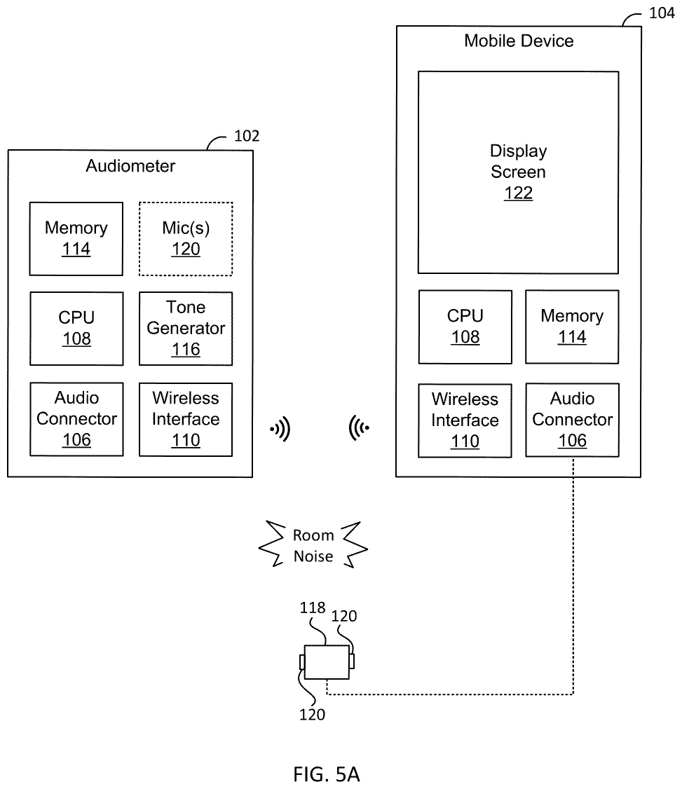

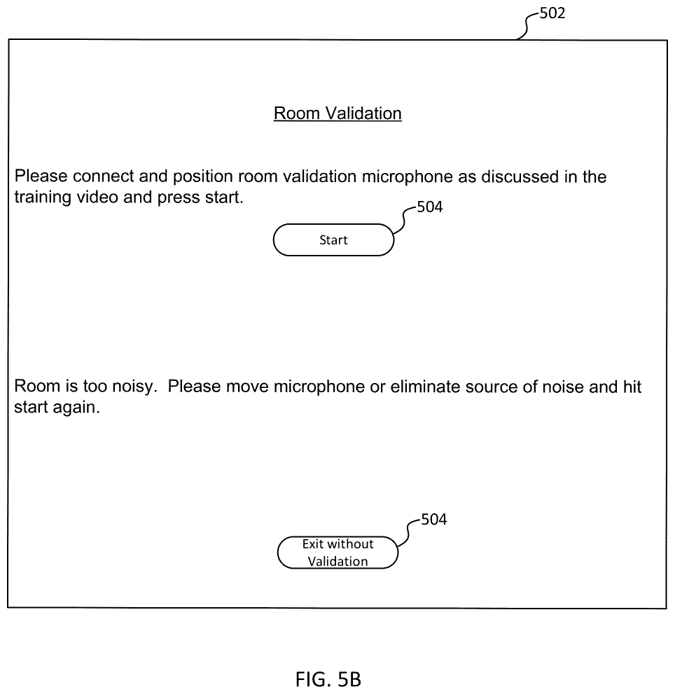

FIG. 5A illustrates a hardware configuration for confirming that the room in which the audiometric test is to be administered has a set of background sound pressure levels that conform to a predetermined standard. Determining the set of background sound pressure levels may be performed, in one embodiment, using the microphone(s) 120 associated with the calibrator 118. Alternatively, the audiometer 102, itself, and/or the mobile device 104 may have microphones that may be used for this purpose.

Sound captured by the microphone(s) 120, which includes ambient noise in the room, may be conveyed to the mobile device 104 via the audio connector 106 (or wirelessly) where it is deconstructed using, e.g., Fourier analysis, and evaluated at each of the octave-band center frequencies shown in Table 1. If the sound pressure level for one or more of the octave-band center frequencies exceeds the corresponding maximum sound pressure levels, the room may not be validated. In such a case, the administrator may be prompted by the room validation module 210 to move the system 100 to a quieter environment, remove or deactivate noisy equipment, and/or take other remedial action.

FIG. 5B shows an example user interface 502 for room validation, which may be provided by the room validation module 210. The user interface 502 may include a control 504 for starting the room validation once the equipment (e.g., microphone) has been positioned. If the room is too noisy, the test administrator will be notified and given an opportunity to rectify the situation. If the room meets the prescribed standard, the system may automatically proceed to equipment validation, as described hereafter. In some embodiments, the user interface 502 may include a control 504 to exit without validation. However, as noted above, without room validation, the audiometric test might be blocked from proceeding in some embodiments.

Returning to FIG. 4A, the method 400 continues by determining 406 that the calibration of the audiometer has been validated at least once on a day (or other set time period) in which the audiometric test is to be administered. Typically, audiometric testing equipment is calibrated at regular intervals, such that the tones generated by the tone generator 116 and reproduced by the headset 112 have frequencies and sound pressure levels that conform to a predetermined standard. However, between these intervals, it is possible that the audiometer 102 and/or the headset 112 may fall out of calibration.

FIG. 6A illustrates a hardware configuration for calibrating the audiometer 102 and headset 112 using the calibrator 118 when the calibration has not been validated within the day (or other time period) in which the audiometric test is to be conducted. As shown, the calibrator 118 may include left and right microphones 120 in one embodiment, which correspond to the left and right drivers of the headset 112, respectively. In some embodiments, the housing of the calibrator 118 has a width that approximates the width of a human head. The headset 112 may be physically placed such that the left driver of the headset 112 is in proximity to left microphone 120 and the right driver of the headset 112 is in proximity to right microphone 120. In some embodiments, the left and right microphones 120 may slightly protrude from the calibrator 118 and be covered by the left and right drivers of the headset 112 in the manner of human ears, replicating the experience of a human wearing the headset 112.

During calibration, the mobile device 104 controls the audiometer 102 in the same manner as an audiometric test (as described more fully below) to generate a series of test tones at various frequencies in each of the left and right drivers of the headset 112. In response, the left microphone 120 and the right microphone 120 of the audiometer 102 will sample the sound produced by the audiometer 102 and output by the headset 112 to determine whether the sampled tones deviate from the test tones by no more than a threshold decibel level. In one embodiment, the threshold decibel level is 10 dB, and the plurality of test tones may be selected from 500 Hz, 1000 Hz, 2000 Hz, 3000 Hz, 4000 Hz, 6000 Hz, and 8000 Hz, which correspond to the tones typically used during audiometric testing.

As in the case of room validation, sound captured by the microphones 120 may be conveyed in analog or digital format to the mobile device 104, where the waveform (frequency and amplitude) of each sampled test tone is analyzed. If the sound pressure level (amplitude) for any test tone differs by more than 10 dB from the expected level, the audiometer 102 and headset 112 may fail validation and the administrator conducting the test may be informed via the display screen 122 so that remedial action may be taken, including, but not limited to, checking connections and/or replacing audiometer 102 and/or the headset 112.

In some embodiments, the audiometer 102 and headset 112 may need to be calibrated, such that the headset 112 outputs a louder (or softer) volume at the frequency where validation failed. If the louder (or softer) tone resolves the issue, the mobile device 104 and/or the audiometer 102 may store the amount of the increase (or decrease) in volume in an equalization table in the memory 114 that will be utilized during the subsequent audiometric test. If the calibration cannot be accomplished, the administrator may be prompted to take more drastic remedial action, such as replacing the headset 112 and/or the audiometer 102.

In some embodiments, a calibrator 118 may not be used. For example, the drivers of the headset 112 may be brought in proximity to one or more microphones 120 within the audiometer 102 or the mobile device 104. In other embodiments, the mobile device 104 may be coupled via wires (or wirelessly) to microphones 120 that may be individually placed in proximity (e.g., inserted into) to the left and right drivers of the headset 112.

In an alternative embodiment or as an additional aspect of validating the calibration of the audiometer 102 and headset 112, a user interface 602 as shown in FIG. 6B may be generated by the equipment validation module 212. The user interface 602 may instruct the administrator to activate a control 604 to play a tone at a predetermined frequency and volume. If the volume is too soft or uncomfortably loud, the user may be allowed to move a slider 606 to select a comfortable volume.

Once the volume has been set, the user may activate a control 608 (e.g., “My volume is set”) which causes a set of tones to be played during which the user may listen for any pops, crackles, or other unwanted noises, which may be indicative of a bad physical connection between the headset 112 and the audiometer 102 and/or interference with a wireless connection. If no unwanted sounds are heard, the user may activate a control 610 to indicate that validation is complete. Alternatively, the user may activate a different control 612 to exit without validation (which may prevent audiometric testing from proceeding).

Returning to FIG. 4A, the method 400 may continue by determining 410 whether the room and equipment have been properly validated. If not, the method 400 may return to step 404 and/or step 406, depending on what type of validation is required. If both types of validation have occurred, the method may continue with step 302 shown in FIG. 3A, by which a subject may register and take the audiometric test.

Data regarding room validation, equipment validation, instruction of the subject and/or administrator, and reporting (including a spoken or otherwise delivered explanation to the subject) may be logged in the memory 114 or another suitable location for subsequent validation of the audiometric test by a test administrator, employer, or third party (e.g., regulatory or standards agency).

The system 100 described above has many advantages over conventional approaches. The audiometer 102 and mobile device 104 may be small and light, making it possible to easily transport the system 100 to different testing sites. Furthermore, the system 100 uses relatively inexpensive components and may be easily repaired if either the audiometer 102 or the mobile device 104 is damaged. The system 100 produces valid audiometric tests by requiring room validation, audiometer/headset validation, and/or proper instruction of the subject, before the audiometric test may commence, providing the ability to certify that an audiometric test and its accompanying results are valid and correct. Finally, the system ensures that both the subject and the test administrator are properly instructed and that acknowledgment of the instruction, as well as acknowledgement that the subject viewed the test results, is stored for later verification.

Numerous examples are provided herein to enhance understanding of the present disclosure. A specific set of statements is provided as follows.

Statement 1. A system for audiometric testing, comprising: an audiometer including a tone generator in electronic communication with a headset worn by a subject to be tested; and a mobile device in electronic communication with the audiometer, wherein the audiometer and the mobile device are separate devices, and the mobile device includes: a display screen; one or more processors; and a non-transitory machine-readable medium including program code that causes the one or more processors to perform a method comprising: displaying, on the display screen, a training video to the subject; conducting an audiometric test of the subject using the mobile device to control the audiometer; displaying, on the display screen, results of the audiometric test; electronically comparing the results of the audiometric test with a baseline test for the subject; displaying, on the display screen, a notification of a hearing shift if the results of the audiometric test differ from the baseline test by more than a predetermined threshold; and receiving, via the mobile device, an electronic acknowledgment from the subject that the subject has viewed the training video, the results of the audiometric test, and the notification of the hearing shift, if any.

Statement 2. The system of statement 1, wherein the electronic acknowledgment comprises an electronic signature.

Statement 3. The system of statements 1-2, wherein displaying the training video comprises preventing the training video from being skipped.

Statement 4. The system of statements 1-3, wherein the method further comprises receiving confirmation from the subject that the training video has been viewed, and wherein conducting the audiometric test is prevented until the confirmation from the subject has been received.

Statement 5. The system of statements 1-4, wherein receiving the confirmation from the subject that the training video has been viewed comprises using focus tracking to determine that the subject watched the training video.

Statement 6. The system of statements 1-5, wherein the method further comprises, prior to conducting the audiometric test: confirming that a room in which the audiometric test is to be administered has a set of background sound pressure levels that conform to a predetermined standard; determining that a calibration of the audiometer has been validated at least once on a day in which the audiometric test is to be administered; and conducting the audiometric test once it is confirmed that the room in which the audiometric test is to be administered has the set of background sound pressure levels that conform to the predetermined standard and it is determined that the calibration of the audiometer has been validated at least once on the day in which the audiometric test is to be administered.

Statement 7. The system of statements 1-6, wherein the predetermined standard for the room includes a plurality of octave-band center frequencies and corresponding maximum sound pressure levels, and wherein confirming comprises confirming the set of background sound pressure levels at each of the plurality of octave-band center frequencies do not exceed the corresponding maximum sound pressure levels.

Statement 8. The system of statement 1-7, wherein confirming that the set of background sound pressure levels at each of the plurality of octave-band center frequencies do not exceed the corresponding maximum sound pressure levels comprises sampling the set of background sound pressure levels at each of the plurality of octave-band center frequencies using the audiometer.

Statement 9. The system of statements 1-8, wherein the plurality of octave-band center frequencies are selected from the group consisting of 500 Hz, 1000 Hz, 2000 Hz, 4000 Hz, and 8000 Hz.

Statement 10. The system of statements 1-9, wherein the corresponding maximum sound pressure levels for the plurality of octave-band center frequencies are selected from the group consisting of 40 dB at 500 Hz, 40 dB at 1000 Hz, 47 dB at 2000 Hz, 57 dB at 4000 Hz, and 62 dB at 8000 Hz.

Statement 11. The system of statements 1-10, wherein determining comprises: in response to the calibration of the audiometer having not been validated at least once on the day in which the audiometric test is to be administered: sampling audio output of the headset at each a plurality of test tones generated by the tone generator; and determining that the audio output of the headset deviates from each of the plurality of test tones by no more than a threshold decibel level.

Statement 12. The system of statements 1-11, wherein determining that the audio output of the headset deviates from each of the plurality of test tones by no more than the threshold decibel level is performed by a calibrator including a housing and two microphones positioned on opposite sides of the housing, wherein the two microphones are configured to be placed in proximity to respective drivers of the headset when the audio output of the headset is being sampled.

Statement 13. The system of statements 1-12, wherein the threshold decibel level is 10 dB, and wherein the plurality of test tones have frequencies selected from the group consisting of 500 Hz, 1000 Hz, 2000 Hz, 3000 Hz, 4000 Hz, 6000 Hz, and 8000 Hz.

Statement 14. The system of statements 1-13, wherein conducting the audiometric test comprises: sending a first structured message from the mobile device to the audiometer instructing the audiometer to generate a tone at a predetermined frequency; receiving a second structured message at the mobile device from the audiometer indicating that the tone has been generated; determining at the mobile device whether the subject has indicated that the tone has been heard within a predetermined time interval; and repeating the steps of sending, receiving, and determining at each of a plurality of predetermined frequencies for each ear of the subject.

Statement 15. The system of statements 1-14, wherein the method further comprises progressively increasing a sound pressure level of a particular tone, in response to the subject failing to indicate that the particular tone has been heard within the predetermined time interval, until the subject has indicated that the tone has been heard within the predetermined time interval.

Statement 16. The system of statements 1-15, wherein the plurality of predetermined frequencies are selected from the group consisting of 500 Hz, 1000 Hz, 2000 Hz, 3000 Hz, 4000 Hz, 6000 Hz, and 8000 Hz, and wherein conducting the audiometric test comprises conducting the audiometric test using a Hughson-Westlake procedure.

Statement 17. The system of statements 1-16, wherein the first structured message and the second structured message comprise Bluetooth packets.

Statement 18. The system of statements 1-17, wherein sending comprises sending the first structured message after a delay within a predetermined delay range.

Statement 19. The system of statements 1-18, wherein displaying the results of the audiometric test comprises generating an audiogram that includes, for each ear of the subject, a graph of hearing threshold levels of the subject for a set of frequencies.

Statement 20. The system of statements 1-19, wherein displaying the training video comprises displaying information regarding: effects of noise on hearing; purposes of hearing protectors; advantages, disadvantages, and attenuation of various types of hearing protectors; selection, fitting, use, and care of the hearing protectors; purposes of audiometric testing, and audiometric testing procedures.

Statement 21. A non-transitory machine-readable medium comprising instructions that, when executed by one or more processors, cause the one or more processors to perform a method for audiometric testing, comprising: displaying, on a mobile device, a training video to a subject to be tested; conducting an audiometric test of the subject using the mobile device to control an audiometer in wireless communication with the mobile device, wherein the audiometer is separate from the mobile device and includes a tone generator in electronic communication with a headset worn by the subject; displaying, on the mobile device, results of the audiometric test; electronically comparing the results of the audiometric test with a baseline test for the subject; displaying, on the mobile device, a notification of a hearing shift if the results of the audiometric test differ from the baseline test by more than a predetermined threshold; and receiving, via the mobile device, an electronic acknowledgment from the subject that the subject has viewed the training video, the results of the audiometric test, and the notification of the hearing shift, if any.

Statement 22. The non-transitory machine-readable medium of statement 21, wherein the electronic acknowledgment comprises an electronic signature.

Statement 23. The non-transitory machine-readable medium of statements 21-22, wherein displaying the training video comprises preventing the training video from being skipped.

Statement 24. The non-transitory machine-readable medium of statements 21-23, further comprising receiving confirmation from the subject that the training video has been viewed, and wherein conducting the audiometric test is prevented until the confirmation from the subject has been received.

Statement 25. The non-transitory machine-readable medium of statements 21-24, wherein receiving the confirmation from the subject that the training video has been viewed comprises using focus tracking to determine that the subject watched the training video.

Statement 26. The non-transitory machine-readable medium of statements 21-25, further comprising, prior to conducting the audiometric test: confirming that a room in which the audiometric test is to be administered has a set of background sound pressure levels that conform to a predetermined standard; determining that a calibration of the audiometer has been validated at least once on a day in which the audiometric test is to be administered; and conducting the audiometric test once it is confirmed that the room in which the audiometric test is to be administered has the set of background sound pressure levels that conform to the predetermined standard and it is determined that the calibration of the audiometer has been validated at least once on the day in which the audiometric test is to be administered.

Statement 27. The non-transitory machine-readable medium of statements 21-26, wherein the predetermined standard for the room includes a plurality of octave-band center frequencies and corresponding maximum sound pressure levels, and wherein confirming comprises confirming the set of background sound pressure levels at each of the plurality of octave-band center frequencies do not exceed the corresponding maximum sound pressure levels.

Statement 28. The non-transitory machine-readable medium of statements 21-27, wherein confirming that the set of background sound pressure levels at each of the plurality of octave-band center frequencies do not exceed the corresponding maximum sound pressure levels comprises sampling the set of background sound pressure levels at each of the plurality of octave-band center frequencies using the audiometer.

Statement 29. The non-transitory machine-readable medium of statements 21-28, wherein the plurality of octave-band center frequencies are selected from the group consisting of 500 Hz, 1000 Hz, 2000 Hz, 4000 Hz, and 8000 Hz.

Statement 30. The non-transitory machine-readable medium of statements 21-29, wherein the corresponding maximum sound pressure levels for the plurality of octave-band center frequencies are selected from the group consisting of 40 dB at 500 Hz, 40 dB at 1000 Hz, 47 dB at 2000 Hz, 57 dB at 4000 Hz, and 62 dB at 8000 Hz.

31. The non-transitory machine-readable medium of statements 21-30, wherein determining comprises: in response to the calibration of the audiometer having not been validated at least once on the day in which the audiometric test is to be administered: sampling audio output of the headset at each a plurality of test tones generated by the tone generator; and determining that the audio output of the headset deviates from each of the plurality of test tones by no more than a threshold decibel level.

Statement 32. The non-transitory machine-readable medium of statements 21-31, wherein determining that the audio output of the headset deviates from each of the plurality of test tones by no more than the threshold decibel level is performed by a calibrator including a housing and two microphones positioned on opposite sides of the housing, wherein the two microphones are configured to be placed in proximity to respective drivers of the headset when the audio output of the headset is being sampled.

Statement 33. The non-transitory machine-readable medium of statements 21-32, wherein the threshold decibel level is 10 dB, and wherein the plurality of test tones have frequencies selected from the group consisting of 500 Hz, 1000 Hz, 2000 Hz, 3000 Hz, 4000 Hz, 6000 Hz, and 8000 Hz.

Statement 34. The non-transitory machine-readable medium of statements 21-33, wherein conducting the audiometric test comprises: sending a first structured message from the mobile device to the audiometer instructing the audiometer to generate a tone at a predetermined frequency; receiving a second structured message at the mobile device from the audiometer indicating that the tone has been generated; determining at the mobile device whether the subject has indicated that the tone has been heard within a predetermined time interval; and repeating the steps of sending, receiving, and determining at each of a plurality of predetermined frequencies for each ear of the subject.

Statement 35. The non-transitory machine-readable medium of statements 21-34, further comprising progressively increasing a sound pressure level of a particular tone, in response to the subject failing to indicate that the particular tone has been heard within the predetermined time interval, until the subject has indicated that the tone has been heard within the predetermined time interval.

Statement 36. The non-transitory machine-readable medium of statements 21-35, wherein the plurality of predetermined frequencies are selected from the group consisting of 500 Hz, 1000 Hz, 2000 Hz, 3000 Hz, 4000 Hz, 6000 Hz, and 8000 Hz, and wherein conducting the audiometric test comprises conducting the audiometric test using a Hughson-Westlake procedure.

Statement 37. The non-transitory machine-readable medium of statements 21-36, wherein the first structured message and the second structured message comprise Bluetooth packets.

Statement 38. The non-transitory machine-readable medium of statements 21-37, wherein sending comprises sending the first structured message after a delay within a predetermined delay range.

Statement 39. The non-transitory machine-readable medium of statements 21-38, wherein displaying the results of the audiometric test comprises generating an audiogram that includes, for each ear of the subject, a graph of hearing threshold levels of the subject for a set of frequencies.

Statement 40. The non-transitory machine-readable medium of statements 21-39, wherein displaying the training video comprises displaying information regarding: effects of noise on hearing; purposes of hearing protectors; advantages, disadvantages, and attenuation of various types of hearing protectors; selection, fitting, use, and care of the hearing protectors; purposes of audiometric testing, and audiometric testing procedures.

Statement 41. A computer-implemented method comprising: displaying, on a mobile device, a training video to a subject to be tested; conducting an audiometric test of the subject using the mobile device to control an audiometer in wireless communication with the mobile device, wherein the audiometer is separate from the mobile device and includes a tone generator in electronic communication with a headset worn by the subject; displaying, on the mobile device, results of the audiometric test; electronically comparing the results of the audiometric test with a baseline test for the subject; displaying, on the mobile device, a notification of a hearing shift if the results of the audiometric test differ from the baseline test by more than a predetermined threshold; and receiving, via the mobile device, an electronic acknowledgment from the subject that the subject has viewed the training video, the results of the audiometric test, and the notification of the hearing shift, if any.

As used herein, the articles “a” and “an” refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

The present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

The systems and methods described herein can be implemented in hardware, software, firmware, or combinations of hardware, software and/or firmware. In some examples, systems described in this specification may be implemented using a non-transitory machine-readable medium storing machine-executable instructions (e.g., program code) that when executed by one or more processors of a computer cause the computer to perform operations (e.g., methods, processes). Machine-readable media suitable for implementing the control systems described in this specification include non-transitory machine-readable media, such as disk memory devices, chip memory devices, programmable logic devices, random access memory (RAM), read only memory (ROM), optical read/write memory, cache memory, magnetic read/write memory, flash memory, and application-specific integrated circuits. In addition, a machine-readable medium that implements a control system described in this specification may be located on a single device or computing platform or may be distributed across multiple devices or computing platforms.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

What is claimed is:

1. A system for audiometric testing, comprising:
an audiometer including a tone generator in electronic communication with a headset worn by a subject to be tested; and
a mobile device in electronic communication with the audiometer, wherein the audiometer and the mobile device are separate devices, and the mobile device includes:
a display screen;
one or more processors; and
a non-transitory machine-readable medium including program code that causes the one or more processors to perform a method comprising:
displaying, on the display screen, a training video to the subject;
conducting an audiometric test of the subject using the mobile device to control the audiometer;
displaying, on the display screen, results of the audiometric test;
electronically comparing the results of the audiometric test with a baseline test for the subject;
displaying, on the display screen, a notification of a hearing shift if the results of the audiometric test differ from the baseline test by more than a predetermined threshold; and
receiving, via the mobile device, an electronic acknowledgment from the subject that the subject has viewed the training video, the results of the audiometric test, and the notification of the hearing shift, if any.

2. The system of claim 1, wherein the electronic acknowledgment comprises an electronic signature.

3. The system of claim 1, wherein displaying the training video comprises preventing the training video from being skipped.

4. The system of claim 1, wherein the method further comprises receiving confirmation from the subject that the training video has been viewed, and wherein conducting the audiometric test is prevented until the confirmation from the subject has been received.

5. The system of claim 4, wherein receiving the confirmation from the subject that the training video has been viewed comprises using focus tracking to determine that the subject watched the training video.

6. The system of claim 1, wherein the method further comprises, prior to conducting the audiometric test:
confirming that a room in which the audiometric test is to be administered has a set of background sound pressure levels that conform to a predetermined standard;
determining that a calibration of the audiometer has been validated at least once on a day in which the audiometric test is to be administered; and
conducting the audiometric test once it is confirmed that the room in which the audiometric test is to be administered has the set of background sound pressure levels that conform to the predetermined standard and it is determined that the calibration of the audiometer has been validated at least once on the day in which the audiometric test is to be administered.

7. The system of claim 6, wherein the predetermined standard for the room includes a plurality of octave-band center frequencies and corresponding maximum sound pressure levels, and wherein confirming comprises confirming the set of background sound pressure levels at each of the plurality of octave-band center frequencies do not exceed the corresponding maximum sound pressure levels.

8. The system of claim 7, wherein confirming that the set of background sound pressure levels at each of the plurality of octave-band center frequencies do not exceed the corresponding maximum sound pressure levels comprises sampling the set of background sound pressure levels at each of the plurality of octave-band center frequencies using the audiometer.

9. The system of claim 7, wherein the plurality of octave-band center frequencies are selected from the group consisting of 500 Hz, 1000 Hz, 2000 Hz, 4000 Hz, and 8000 Hz.

10. The system of claim 7, wherein the corresponding maximum sound pressure levels for the plurality of octave-band center frequencies are selected from the group consisting of 40 dB at 500 Hz, 40 dB at 1000 Hz, 47 dB at 2000 Hz, 57 dB at 4000 Hz, and 62 dB at 8000 Hz.

11. The system of claim 6, wherein determining comprises:
in response to the calibration of the audiometer having not been validated at least once on the day in which the audiometric test is to be administered:
sampling audio output of the headset at each a plurality of test tones generated by the tone generator; and
determining that the audio output of the headset deviates from each of the plurality of test tones by no more than a threshold decibel level.

12. The system of claim 11, wherein determining that the audio output of the headset deviates from each of the plurality of test tones by no more than the threshold decibel level is performed by a calibrator including a housing and two microphones positioned on opposite sides of the housing, wherein the two microphones are configured to be placed in proximity to respective drivers of the headset when the audio output of the headset is being sampled.

13. The system of claim 11, wherein the threshold decibel level is 10 dB, and wherein the plurality of test tones have frequencies selected from the group consisting of 500 Hz, 1000 Hz, 2000 Hz, 3000 Hz, 4000 Hz, 6000 Hz, and 8000 Hz.

14. The system of claim 1, wherein conducting the audiometric test comprises:
sending a first structured message from the mobile device to the audiometer instructing the audiometer to generate a tone at a predetermined frequency;
receiving a second structured message at the mobile device from the audiometer indicating that the tone has been generated;
determining at the mobile device whether the subject has indicated that the tone has been heard within a predetermined time interval; and
repeating the steps of sending, receiving, and determining at each of a plurality of predetermined frequencies for each ear of the subject.

15. The system of claim 14, wherein the method further comprises progressively increasing a sound pressure level of a particular tone, in response to the subject failing to indicate that the particular tone has been heard within the predetermined time interval, until the subject has indicated that the tone has been heard within the predetermined time interval.

16. The system of claim 15, wherein the plurality of predetermined frequencies are selected from the group consisting of 500 Hz, 1000 Hz, 2000 Hz, 3000 Hz, 4000 Hz, 6000 Hz, and 8000 Hz, and wherein conducting the audiometric test comprises conducting the audiometric test using a Hughson-Westlake procedure.

17. The system of claim 15, wherein the first structured message and the second structured message comprise Bluetooth packets.

18. The system of claim 15, wherein sending comprises sending the first structured message after a delay within a predetermined delay range.

19. The system of claim 1, wherein displaying the results of the audiometric test comprises generating an audiogram that includes, for each ear of the subject, a graph of hearing threshold levels of the subject for a set of frequencies.

20. The system of claim 1, wherein displaying the training video comprises displaying information regarding:
effects of noise on hearing;
purposes of hearing protectors;
advantages, disadvantages, and attenuation of various types of hearing protectors;
selection, fitting, use, and care of the hearing protectors;
purposes of audiometric testing, and
audiometric testing procedures.

21. A non-transitory machine-readable medium comprising instructions that, when executed by one or more processors, cause the one or more processors to perform a method for audiometric testing, comprising:
displaying, on a mobile device, a training video to a subject to be tested;
conducting an audiometric test of the subject using the mobile device to control an audiometer in wireless communication with the mobile device, wherein the audiometer is separate from the mobile device and includes a tone generator in electronic communication with a headset worn by the subject;
displaying, on the mobile device, results of the audiometric test;
electronically comparing the results of the audiometric test with a baseline test for the subject;
displaying, on the mobile device, a notification of a hearing shift if the results of the audiometric test differ from the baseline test by more than a predetermined threshold; and
receiving, via the mobile device, an electronic acknowledgment from the subject that the subject has viewed the training video, the results of the audiometric test, and the notification of the hearing shift, if any.

22. The non-transitory machine-readable medium of claim 21, wherein the electronic acknowledgment comprises an electronic signature.

23. The non-transitory machine-readable medium of claim 21, wherein displaying the training video comprises preventing the training video from being skipped.

24. The non-transitory machine-readable medium of claim 21, further comprising receiving confirmation from the subject that the training video has been viewed, and wherein conducting the audiometric test is prevented until the confirmation from the subject has been received.

25. The non-transitory machine-readable medium of claim 24, wherein receiving the confirmation from the subject that the training video has been viewed comprises using focus tracking to determine that the subject watched the training video.

26. The non-transitory machine-readable medium of claim 21, further comprising, prior to conducting the audiometric test:
confirming that a room in which the audiometric test is to be administered has a set of background sound pressure levels that conform to a predetermined standard;
determining that a calibration of the audiometer has been validated at least once on a day in which the audiometric test is to be administered; and
conducting the audiometric test once it is confirmed that the room in which the audiometric test is to be administered has the set of background sound pressure levels that conform to the predetermined standard and it is determined that the calibration of the audiometer has been validated at least once on the day in which the audiometric test is to be administered.

27. The non-transitory machine-readable medium of claim 26, wherein the predetermined standard for the room includes a plurality of octave-band center frequencies and corresponding maximum sound pressure levels, and wherein confirming comprises confirming the set of background sound pressure levels at each of the plurality of octave-band center frequencies do not exceed the corresponding maximum sound pressure levels.

28. The non-transitory machine-readable medium of claim 27, wherein confirming that the set of background sound pressure levels at each of the plurality of octave-band center frequencies do not exceed the corresponding maximum sound pressure levels comprises sampling the set of background sound pressure levels at each of the plurality of octave-band center frequencies using the audiometer.

29. The non-transitory machine-readable medium of claim 27, wherein the plurality of octave-band center frequencies are selected from the group consisting of 500 Hz, 1000 Hz, 2000 Hz, 4000 Hz, and 8000 Hz.

30. The non-transitory machine-readable medium of claim 27, wherein the corresponding maximum sound pressure levels for the plurality of octave-band center frequencies are selected from the group consisting of 40 dB at 500 Hz, 40 dB at 1000 Hz, 47 dB at 2000 Hz, 57 dB at 4000 Hz, and 62 dB at 8000 Hz.

31. The non-transitory machine-readable medium of claim 26, wherein determining comprises:
in response to the calibration of the audiometer having not been validated at least once on the day in which the audiometric test is to be administered:
sampling audio output of the headset at each a plurality of test tones generated by the tone generator; and
determining that the audio output of the headset deviates from each of the plurality of test tones by no more than a threshold decibel level.

32. The non-transitory machine-readable medium of claim 31, wherein determining that the audio output of the headset deviates from each of the plurality of test tones by no more than the threshold decibel level is performed by a calibrator including a housing and two microphones positioned on opposite sides of the housing, wherein the two microphones are configured to be placed in proximity to respective drivers of the headset when the audio output of the headset is being sampled.

33. The non-transitory machine-readable medium of claim 31, wherein the threshold decibel level is 10 dB, and wherein the plurality of test tones have frequencies selected from the group consisting of 500 Hz, 1000 Hz, 2000 Hz, 3000 Hz, 4000 Hz, 6000 Hz, and 8000 Hz.

34. The non-transitory machine-readable medium of claim 21, wherein conducting the audiometric test comprises:
sending a first structured message from the mobile device to the audiometer instructing the audiometer to generate a tone at a predetermined frequency;
receiving a second structured message at the mobile device from the audiometer indicating that the tone has been generated;
determining at the mobile device whether the subject has indicated that the tone has been heard within a predetermined time interval; and
repeating the steps of sending, receiving, and determining at each of a plurality of predetermined frequencies for each ear of the subject.

35. The non-transitory machine-readable medium of claim 34, further comprising progressively increasing a sound pressure level of a particular tone, in response to the subject failing to indicate that the particular tone has been heard within the predetermined time interval, until the subject has indicated that the tone has been heard within the predetermined time interval.

36. The non-transitory machine-readable medium of claim 35, wherein the plurality of predetermined frequencies are selected from the group consisting of 500 Hz, 1000 Hz, 2000 Hz, 3000 Hz, 4000 Hz, 6000 Hz, and 8000 Hz, and wherein conducting the audiometric test comprises conducting the audiometric test using a Hughson-Westlake procedure.

37. The non-transitory machine-readable medium of claim 35, wherein the first structured message and the second structured message comprise Bluetooth packets.

38. The non-transitory machine-readable medium of claim 35, wherein sending comprises sending the first structured message after a delay within a predetermined delay range.

39. The non-transitory machine-readable medium of claim 21, wherein displaying the results of the audiometric test comprises generating an audiogram that includes, for each ear of the subject, a graph of hearing threshold levels of the subject for a set of frequencies.

40. The non-transitory machine-readable medium of claim 21, wherein displaying the training video comprises displaying information regarding:
effects of noise on hearing;
purposes of hearing protectors;
advantages, disadvantages, and attenuation of various types of hearing protectors;
selection, fitting, use, and care of the hearing protectors;
purposes of audiometric testing, and
audiometric testing procedures.

41. A computer-implemented method comprising:
displaying, on a mobile device, a training video to a subject to be tested;
conducting an audiometric test of the subject using the mobile device to control an audiometer in wireless communication with the mobile device, wherein the audiometer is separate from the mobile device and includes a tone generator in electronic communication with a headset worn by the subject;
displaying, on the mobile device, results of the audiometric test;
electronically comparing the results of the audiometric test with a baseline test for the subject;
displaying, on the mobile device, a notification of a hearing shift if the results of the audiometric test differ from the baseline test by more than a predetermined threshold; and
receiving, via the mobile device, an electronic acknowledgment from the subject that the subject has viewed the training video, the results of the audiometric test, and the notification of the hearing shift, if any.
